(19) United States
(12) Patent Application Publication
(10) Pub. No.: US 2019/0198138 A1
(43) Pub. Date: Jun. 27, 2019

(54) Automatic Expansion of Medically Relevant Summarization Templates Using Semantic Expansion

(71) Applicant: International Business Machines Corporation, Armonk, NY (US)

(72) Inventors: Tyler Baldwin, Union City, CA (US); Ashutosh Jadhav, Santa Clara, CA (US); Chaitanya Shivade, San Jose, CA (US); Tanveer F. Syeda-Mahmood, Cupertino, CA (US); Joy Wu, Mountain View, CA (US)

(21) Appl. No.: 15/854,179

(22) Filed: Dec. 26, 2017

(51) Int. Cl. G16H 10/60; G06F 17/30

(52) U.S. Cl. CPC G16H 10/60; G16H 10/20; G06F 17/30719

(57) ABSTRACT

Mechanisms are provided to implement a medical information summarization engine (MISE). The MISE receives input specifying a summarization template, wherein the summarization template specifies terms or concepts of interest to a medical professional when making a medical decision regarding a patient. The MISE expands the summarization template based on related concepts or related terms related to the terms or concepts of interest specified in the summarization template. The MISE processes an EMR of the patient based on the expanded summarization template to extract patient information corresponding to the terms or concepts of interest and the related concepts or related terms. The MISE generates and outputs a holistic summary of the EMR of the patient that summarizes the most salient portions of the patient EMR for use by the medical professional in making the medical decision regarding the patient, based on extracted patient information obtained from processing the patient EMR.

### BACKGROUND

The present application relates generally to an improved data processing apparatus and method and more specifically to mechanisms for automatically expanding medically relevant summarization templates using semantic expansion.

Decision-support systems exist in many different industries where human experts require assistance in retrieving and analyzing information. An example that will is a diagnosis system employed in the healthcare industry. Diagnosis systems can be classified into systems that use structured knowledge, systems that use unstructured knowledge, and systems that use clinical decision formulas, rules, trees, or algorithms. The earliest diagnosis systems used structured knowledge or classical, manually constructed knowledge bases. The Internist-I system developed in the 1970s uses disease-finding relations and disease-disease relations. The MYCIN system for diagnosing infectious diseases, also developed in the 1970s, uses structured knowledge in the form of production rules, stating that if certain facts are true, then one can conclude certain other facts with a given certainty factor. DXplain, developed starting in the 1980s, uses structured knowledge similar to that of Internist-I, but adds a hierarchical lexicon of findings.

Iliad, developed starting in the 1990s, adds more sophisticated probabilistic reasoning where each disease has an associated a priori probability of the disease (in the population for which Iliad was designed), and a list of findings along with the fraction of patients with the disease who have the finding (sensitivity), and the fraction of patients without the disease who have the finding (1-specificity).

In 2000, diagnosis systems using unstructured knowledge started to appear. These systems use some structuring of knowledge such as, for example, entities such as findings and disorders being tagged in documents to facilitate retrieval. ISABEL, for example, uses Autonomy information retrieval software and a database of medical textbooks to retrieve appropriate diagnoses given input findings. Autonomy Auminence uses the Autonomy technology to retrieve diagnoses given findings and organizes the diagnoses by body system. First CONSULT allows one to search a large collection of medical books, journals, and guidelines by chief complaints and age group to arrive at possible diagnoses. PEPID DDX is a diagnosis generator based on PEPID's independent clinical content.

Clinical decision rules have been developed for a number of medical disorders, and computer systems have been developed to help practitioners and patients apply these rules. The Acute Cardiac Ischemia Time-Insensitive Predictive Instrument (ACI-TIPI) takes clinical and ECG features as input and produces probability of acute cardiac ischemia as output to assist with triage of patients with chest pain or other symptoms suggestive of acute cardiac ischemia. ACI-TIPI is incorporated into many commercial heart monitors/defibrillators. The CaseWalker system uses a four-item questionnaire to diagnose major depressive disorder. The PKC Advisor provides guidance on 98 patient problems such as abdominal pain and vomiting.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method, in a data processing system comprising a processor and a memory, the memory comprising instructions that are executed by the processor to specifically configure the processor to implement a medical information summarization engine (MISE). The method comprises receiving, by the MISE executing in the data processing system, input specifying a summarization template, wherein the summarization template specifies terms or concepts of interest to a medical professional when making a medical decision regarding a patient. The method also comprises expanding, by the MISE, the summarization template based on related concepts or related terms that are related to the terms or concepts of interest specified in the summarization template, to thereby generate an expanded summarization template, wherein the expansion operation expands the summarization template based on a traversal of a medical knowledge base of the related terms or relate concepts. In addition, in response to receiving electronic medical records (EMR) for a patient, the method comprises processing, by the MISE, the EMR of the patient based on the expanded summarization template to extract patient information corresponding to the terms or concepts of interest and the related concepts or related terms. Further, the method comprises generating and outputting, by the MISE, a holistic summary of the EMR of the patient that summarizes the most salient portions of the patient EMR for use by the medical professional in making the medical decision regarding the patient, based on extracted patient information obtained from processing the patient EMR based on the expanded summarization template.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE SEVERAL VIEWS OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 depicts a schematic diagram of one illustrative embodiment of a cognitive system in a computer network;

FIG. 2 is a block diagram of an example data processing system in which aspects of the illustrative embodiments are implemented;

FIG. 3 is an example diagram illustrating an interaction of elements of a healthcare cognitive system in accordance with one illustrative embodiment;

FIG. 4 depicts a functional block diagram of operations performed by a medical information summarization mechanism in automatically summarizing patient data using medically relevant summarization templates in accordance with an illustrative embodiment; and

FIG. 5 depicts a functional block diagram of operations performed by a medical information summarization mechanism in automatically expand medically relevant summarization templates using semantic expansion in accordance with an illustrative embodiment.

### DETAILED DESCRIPTION

The strengths of current cognitive systems, such as current medical diagnosis, patient health management, patient treatment recommendation systems, law enforcement investigation systems, and other decision support systems, are that they can provide insights that improve the decision making performed by human beings. For example, in the medical context, such cognitive systems may improve medical practitioners' diagnostic hypotheses, can help medical practitioners avoid missing important diagnoses, and can assist medical practitioners with determining appropriate treatments for specific diseases. However, current systems still suffer from significant drawbacks which should be addressed in order to make such systems more accurate and usable for a variety of applications as well as more representative of the way in which human beings make decisions, such as diagnosing and treating patients. In particular, one drawback of current systems is that patient electronic medical records (EMRs) usually contain very detailed information and are a source of a large amount of patient data for a patient, leading to an information overload condition for the medical professional. It is difficult for a medical professional to identify the most relevant information for making a medical decision when presented with so much patient EMR information. Reaching actionable information within such a large collection of data is hard to achieve and is time consuming for the medical professional leading to difficulties in obtaining a holistic summary of the patient.

Thus, it would be beneficial to have a mechanism for summarizing the most medically relevant information pertinent to the needs of the particular medical professional and the medical decisions being made. The illustrative embodiments provide mechanisms that automatically summarize patient data using medically relevant summarization templates. That is, the mechanisms distill important information from a patient's EMRs using an expert verified summarization template. The mechanisms create a summary template that describes key information identified by the medical professional to be fetched from the patient's EMRs. The mechanisms aggregate redundant pieces of information for conciseness and extract patient information from the patient's EMRs that matches the summarization template. The mechanisms then rank the extracted patient information from the patient's EMRs in light of those matches and generate a patient EMR summary output that summarizes the most salient portions of the patient's EMRs for use by the medical professional in making a medical decision regarding the patient, based on the ranking of the patient information.

Additionally, the illustrative embodiments provide mechanisms that automatically expand medically relevant summarization templates using semantic expansion. In the creation of the summary template that describes key information identified by the medical professional to be fetched from the patient's EMRs, the medical professional may request or indicate that the summary template be expanded to include semantically relevant terms to those identified by the medical professional. Thus, the mechanisms identify the seed concepts and terms provided by the medical professional. The mechanisms expand the seed concepts and terms by identifying medical variants and related concepts based on an ontological hierarchy and a biomedical knowledge graph. In identifying the medical variants and related concepts of the seed concepts and terms; duplicate concepts may be identified. Thus, the mechanisms also mark duplicate concepts in creating a marked-up expanded summarization template. The mechanisms then generate an expanded medically relevant summarization template that is presented to the medical professional prior to summarizing patient data from the patient's EMRs using the marked-up expanded medically relevant summarization templates.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a,” “at least one of,” and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

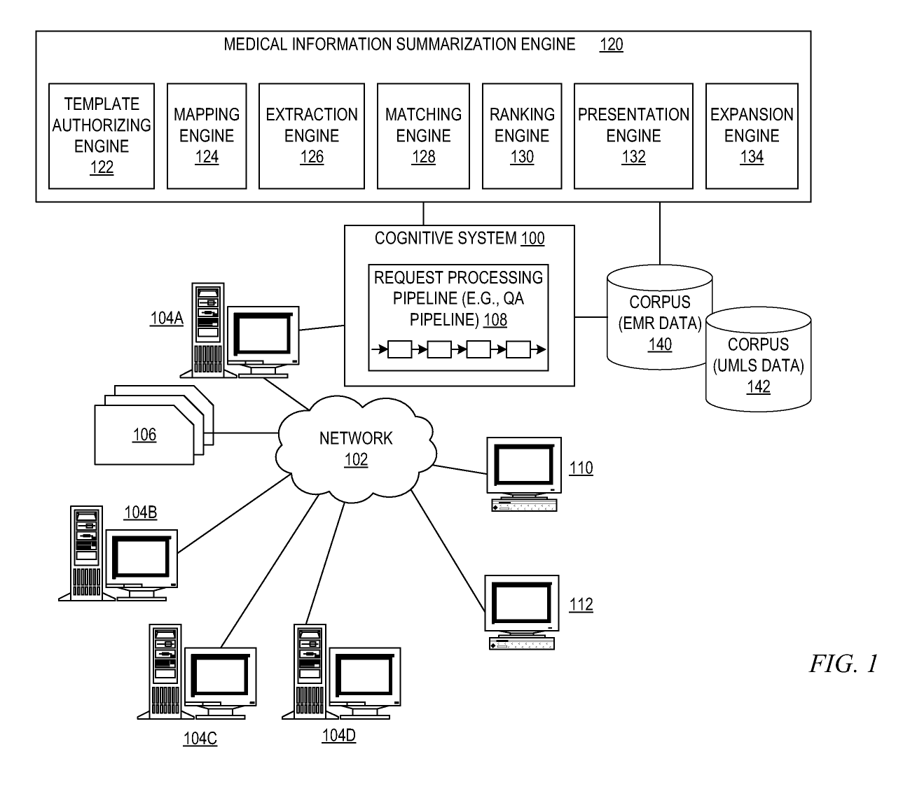

As noted above, the present invention provides mechanisms for automatically summarizing patient data using medically relevant summarization templates and automatically expanding medically relevant summarization templates using semantic expansion. Thus, the illustrative embodiments may be utilized in many different types of data processing environments. In order to provide a context for the description of the specific elements and functionality of the illustrative embodiments, FIGS. 1-3 are provided hereafter as example environments in which aspects of the illustrative embodiments may be implemented. It should be appreciated that FIGS. 1-3 are only examples and are not intended to assert or imply any limitation with regard to the environments in which aspects or embodiments of the present invention may be implemented. Many modifications to the depicted environments may be made without departing from the spirit and scope of the present invention.

FIGS. 1-3 are directed to describing an example cognitive system for automatically summarizing patient data using medically relevant summarization templates and automatically expanding medically relevant summarization templates using semantic expansion which implements a request processing pipeline, request processing methodology, and request processing computer program product with which the mechanisms of the illustrative embodiments are implemented. These requests may be provided as structure or unstructured request messages, natural language questions, or any other suitable format for requesting an operation to be performed by the cognitive system. As described in more detail hereafter, the particular application that is implemented in the cognitive system of the present invention is an application for medical information summarization.

It should be appreciated that the cognitive system, while shown as having a single request processing pipeline in the examples hereafter, may in fact have multiple request processing pipelines. Each request processing pipeline may be separately trained and/or configured to process requests associated with different domains or be configured to perform the same or different analysis on input requests, depending on the desired implementation. For example, in some cases, a first request processing pipeline may be trained to operate on input requests directed to automatically summarizing patient data using medically relevant summarization templates. In other cases, for example, the request processing pipelines may be configured to provide different types of cognitive functions or support different types of applications, such as one request processing pipeline being used for and automatically expanding medically relevant summarization templates using semantic expansion, etc.

Moreover, each request processing pipeline may have its own associated corpus or corpora that they ingest and operate on, e.g., one corpus for patient electronic medical records (EMRs) and another corpus for a knowledge base on related medical terms and medical concepts in the above examples. In some cases, the request processing pipelines may each operate on the same domain of requests but may have different configurations, e.g., different annotators or differently trained annotators, such that different analysis and potential answers are generated. The cognitive system may provide additional logic for routing requests to the appropriate request processing pipeline, such as based on a determined domain of the input request, combining and evaluating final results generated by the processing performed by multiple request processing pipelines, and other control and interaction logic that facilitates the utilization of multiple request processing pipelines.

It should be appreciated that while the present invention will be described in the context of the cognitive system implementing one or more request processing pipelines that operate on a request, the illustrative embodiments are not limited to such. Rather, the mechanisms of the illustrative embodiments may operate on requests that are posed as “questions” or formatted as requests for the cognitive system to perform cognitive operations on a specified set of input data using the associated corpus or corpora and the specific configuration information used to configure the cognitive system. For example, the cognitive system may operate on a natural language question of “What information is there on heart issues that applies to patient P?” as well as the cognitive system operating on a request of “generate a summary of heart issues information for patient P,” or the like. It should be appreciated that the mechanisms of the request processing pipeline may operate on requests in a similar manner to that of input natural language questions with minor modifications. In fact, in some cases, a request may be converted to a natural language question for processing by the request processing pipelines if desired for the particular implementation.

As will be discussed in greater detail hereafter, the illustrative embodiments may be integrated in, augment, and extend the functionality of the request processing pipeline, with regard to automatically summarizing patient data using medically relevant summarization templates and automatically expanding medically relevant summarization templates using semantic expansion.

Thus, it is important to first have an understanding of how cognitive systems implement a request processing pipeline before describing how the mechanisms of the illustrative embodiments are integrated in and augment such cognitive systems and request processing pipeline mechanisms. It should be appreciated that the mechanisms described in FIGS. 1-3 are only examples and are not intended to state or imply any limitation with regard to the type of cognitive system mechanisms with which the illustrative embodiments are implemented. Many modifications to the example cognitive system shown in FIGS. 1-3 may be implemented in various embodiments of the present invention without departing from the spirit and scope of the present invention.

As an overview, a cognitive system is a specialized computer system, or set of computer systems, configured with hardware and/or software logic (in combination with hardware logic upon which the software executes) to emulate human cognitive functions. These cognitive systems apply human-like characteristics to conveying and manipulating ideas which, when combined with the inherent strengths of digital computing, can solve problems with high accuracy and resilience on a large scale. A cognitive system performs one or more computer-implemented cognitive operations that approximate a human thought process as well as enable people and machines to interact in a more natural manner so as to extend and magnify human expertise and cognition. A cognitive system comprises artificial intelligence logic, such as natural language processing (NLP) based logic, for example, and machine learning logic, which may be provided as specialized hardware, software executed on hardware, or any combination of specialized hardware and software executed on hardware. The logic of the cognitive system implements the cognitive operation(s), examples of which include, but are not limited to, question answering, identification of related concepts within different portions of content in a corpus, intelligent search algorithms, such as Internet web page searches, for example, medical diagnostic and treatment recommendations, and other types of recommendation generation, e.g., items of interest to a particular user, potential new contact recommendations, or the like.

IBM Watson™ is an example of one such cognitive system which can process human readable language and identify inferences between text passages with human-like high accuracy at speeds far faster than human beings and on a larger scale. In general, such cognitive systems are able to perform the following functions:
Navigate the complexities of human language and understanding
Ingest and process vast amounts of structured and unstructured data
Generate and evaluate hypothesis
Weigh and evaluate responses that are based only on relevant evidence
Provide situation-specific advice, insights, and guidance
Improve knowledge and learn with each iteration and interaction through machine learning processes
Enable decision making at the point of impact (contextual guidance)
Scale in proportion to the task
Extend and magnify human expertise and cognition
Identify resonating, human-like attributes and traits from natural language
Deduce various language specific or agnostic attributes from natural language
High degree of relevant recollection from data points (images, text, voice) (memorization and recall)
Predict and sense with situational awareness that mimic human cognition based on experiences
Answer questions based on natural language and specific evidence

In one aspect, cognitive systems provide mechanisms for answering requests posed to these cognitive systems using a request processing pipeline and/or process requests which may or may not be posed as natural language questions. The request processing pipeline is an artificial intelligence application executing on data processing hardware that answers requests pertaining to a given subject-matter domain presented in natural language. The request processing pipeline receives inputs from various sources including input over a network, a corpus of electronic documents or other data, data from a content creator, information from one or more content users, and other such inputs from other possible sources of input. Data storage devices store the corpus of data. A content creator creates content in a document for use as part of a corpus of data with the request processing pipeline. The document may include any file, text, article, or source of data for use in the request processing system. For example, a request processing pipeline accesses a body of knowledge about the domain, or subject matter area, e.g., financial domain, medical domain, legal domain, etc., where the body of knowledge (knowledgebase) can be organized in a variety of configurations, e.g., a structured repository of domain-specific information, such as ontologies, or unstructured data related to the domain, or a collection of natural language documents about the domain.

Content users requests to cognitive system which implements the request processing pipeline. The request processing pipeline then answers the requests using the content in the corpus of data by evaluating documents, sections of documents, portions of data in the corpus, or the like. When a process evaluates a given section of a document for semantic content, the process can use a variety of conventions to query such document from the request processing pipeline, e.g., sending the query to the request processing pipeline as a well-formed request which is then interpreted by the request processing pipeline and a response is provided containing one or more answers to the request. Semantic content is content based on the relation between signifiers, such as words, phrases, signs, and symbols, and what they stand for, their denotation, or connotation. In other words, semantic content is content that interprets an expression, such as by using Natural Language Processing.

As will be described in greater detail hereafter, the request processing pipeline receives a request, parses the request to extract the major features of the request, uses the extracted features to formulate queries, and then applies those queries to the corpus of data. Based on the application of the queries to the corpus of data, the request processing pipeline generates a set of hypotheses, or candidate answers to the request, by looking across the corpus of data for portions of the corpus of data that have some potential for containing a valuable response to the request. The request processing pipeline then performs deep analysis on the language of the request and the language used in each of the portions of the corpus of data found during the application of the queries using a variety of reasoning algorithms. There may be hundreds or even thousands of reasoning algorithms applied, each of which performs different analysis, e.g., comparisons, natural language analysis, lexical analysis, or the like, and generates a score. For example, some reasoning algorithms may look at the matching of terms and synonyms within the language of the request and the found portions of the corpus of data. Other reasoning algorithms may look at temporal or spatial features in the language, while others may evaluate the source of the portion of the corpus of data and evaluate its veracity.

The scores obtained from the various reasoning algorithms indicate the extent to which the potential response is inferred by the request based on the specific area of focus of that reasoning algorithm. Each resulting score is then weighted against a statistical model. The statistical model captures how well the reasoning algorithm performed at establishing the inference between two similar passages for a particular domain during the training period of the request processing pipeline. The statistical model is used to summarize a level of confidence that the request processing pipeline has regarding the evidence that the potential response, i.e. candidate answer, is inferred by the request. This process is repeated for each of the candidate answers until the request processing pipeline identifies candidate answers that surface as being significantly stronger than others and thus, generates a final answer, or ranked set of answers, for the request.

As mentioned above, request processing pipeline mechanisms operate by accessing information from a corpus of data or information (also referred to as a corpus of content), analyzing it, and then generating answer results based on the analysis of this data. Accessing information from a corpus of data typically includes: a database query that answers requests about what is in a collection of structured records, and a search that delivers a collection of document links in response to a query against a collection of unstructured data (text, markup language, etc.). Conventional request processing systems are capable of generating answers based on the corpus of data and the request, verifying answers to a collection of requests for the corpus of data, correcting errors in digital text using a corpus of data, and selecting answers to requests from a pool of potential answers, i.e. candidate answers.

Content creators, such as article authors, electronic document creators, web page authors, document database creators, and the like, determine use cases for products, solutions, and services described in such content before writing their content. Consequently, the content creators know what requests the content is intended to answer in a particular topic addressed by the content. Categorizing the requests, such as in terms of roles, type of information, tasks, or the like, associated with the request, in each document of a corpus of data allows the request processing pipeline to more quickly and efficiently identify documents containing content related to a specific query. The content may also answer other requests that the content creator did not contemplate that may be useful to content users. The requests and answers may be verified by the content creator to be contained in the content for a given document. These capabilities contribute to improved accuracy, system performance, machine learning, and confidence of the request processing pipeline. Content creators, automated tools, or the like, annotate or otherwise generate metadata for providing information useable by the QA pipeline to identify these request and answer attributes of the content.

Operating on such content, the request processing pipeline generates answers for requests using a plurality of intensive analysis mechanisms which evaluate the content to identify the most probable answers, i.e. candidate answers, for the request. The most probable answers are output as a ranked listing of candidate answers ranked according to their relative scores or confidence measures calculated during evaluation of the candidate answers, as a single final answer having a highest ranking score or confidence measure, or which is a best match to the request, or a combination of ranked listing and final answer.

FIG. 1 depicts a schematic diagram of one illustrative embodiment of a cognitive system 100 implementing a request processing pipeline 108, which in some embodiments may be a request processing pipeline, in a computer network 102. For purposes of the present description, it will be assumed that the request processing pipeline 108 operates on structured and/or unstructured requests in the form of requests. One example of a request processing operation which may be used in conjunction with the principles described herein is described in U.S. Patent Application Publication No. 2011/0125734, which is herein incorporated by reference in its entirety. The cognitive system 100 is implemented on one or more computing devices 104A-D (comprising one or more processors and one or more memories, and potentially any other computing device elements generally known in the art including buses, storage devices, communication interfaces, and the like) connected to the computer network 102. For purposes of illustration only, FIG. 1 depicts the cognitive system 100 being implemented on computing device 104A only, but as noted above the cognitive system 100 may be distributed across multiple computing devices, such as a plurality of computing devices 104A-D. The network 102 includes multiple computing devices 104A-D, which may operate as server computing devices, and 110-112 which may operate as client computing devices, in communication with each other and with other devices or components via one or more wired and/or wireless data communication links, where each communication link comprises one or more of wires, routers, switches, transmitters, receivers, or the like. In some illustrative embodiments, the cognitive system 100 and network 102 enables request processing functionality for one or more cognitive system users via their respective computing devices 110-112. In other embodiments, the cognitive system 100 and network 102 may provide other types of cognitive operations including, but not limited to, request processing and cognitive response generation which may take many different forms depending upon the desired implementation, e.g., cognitive information retrieval, training/instruction of users, cognitive evaluation of data, or the like. Other embodiments of the cognitive system 100 may be used with components, systems, sub-systems, and/or devices other than those that are depicted herein.

The cognitive system 100 is configured to implement a request processing pipeline 108 that receive inputs from various sources. The requests may be posed in the form of a natural language question, natural language request for information, natural language request for the performance of a cognitive operation, or the like. For example, the cognitive system 100 receives input from the network 102, a corpus or corpora of electronic documents 106, cognitive system users, and/or other data and other possible sources of input. In one embodiment, some or all of the inputs to the cognitive system 100 are routed through the network 102. The various computing devices 104A-D on the network 102 include access points for content creators and cognitive system users. Some of the computing devices 104A-D include devices for a database storing the corpus or corpora of data 106 (which is shown as a separate entity in FIG. 1 for illustrative purposes only). Portions of the corpus or corpora of data 106 may also be provided on one or more other network attached storage devices, in one or more databases, or other computing devices not explicitly shown in FIG. 1. The network 102 includes local network connections and remote connections in various embodiments, such that the cognitive system 100 may operate in environments of any size, including local and global, e.g., the Internet.

In one embodiment, the content creator creates content in a document of the corpus or corpora of data 106 for use as part of a corpus of data with the cognitive system 100. The document includes any file, text, article, or source of data for use in the cognitive system 100. Cognitive system users access the cognitive system 100 via a network connection or an Internet connection to the network 102, and requests to the cognitive system 100 that are answered/processed based on the content in the corpus or corpora of data 106. In one embodiment, the requests are formed using natural language. The cognitive system 100 parses and interprets the request via a pipeline 108, and provides a response to the cognitive system user, e.g., cognitive system user 110, containing one or more answers to the request posed, response to the request, results of processing the request, or the like. In some embodiments, the cognitive system 100 provides a response to users in a ranked list of candidate answers/responses while in other illustrative embodiments, the cognitive system 100 provides a single final answer/response or a combination of a final answer/response and ranked listing of other candidate answers/responses.

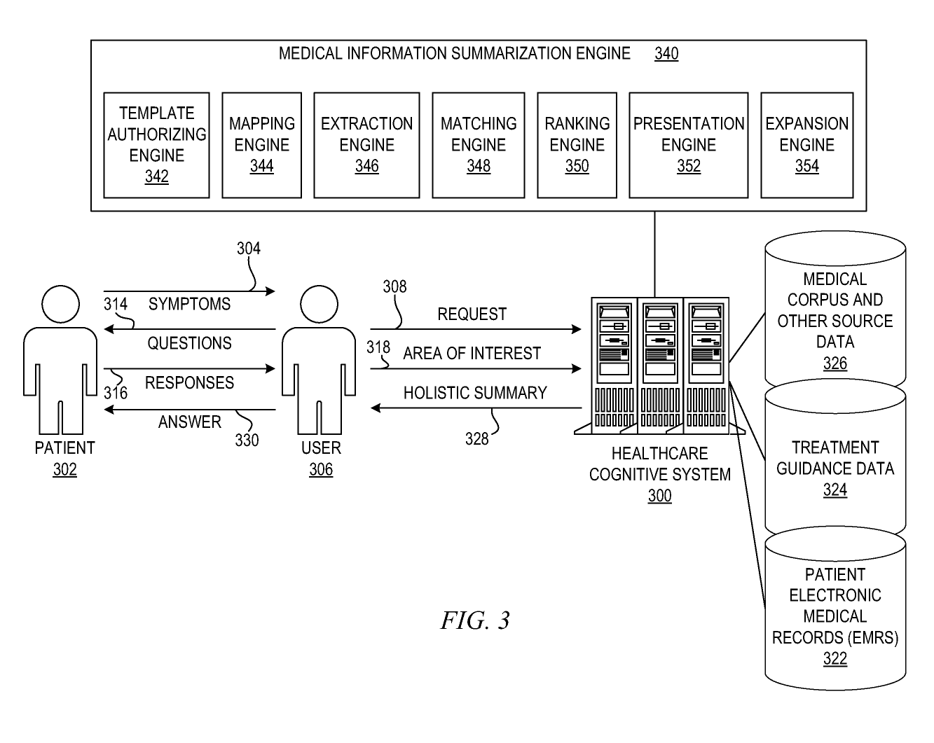

The cognitive system 100 implements the pipeline 108 which comprises a plurality of stages for processing a request based on information obtained from the corpus or corpora of data 106. The pipeline 108 generates answers/responses for the request based on the processing of the request and the corpus or corpora of data 106. The pipeline 108 will be described in greater detail hereafter with regard to FIG. 3.

In some illustrative embodiments, the cognitive system 100 may be the IBM Watson™ cognitive system available from International Business Machines Corporation of Armonk, N.Y., which is augmented with the mechanisms of the illustrative embodiments described hereafter. As outlined previously, a pipeline of the IBM Watson™ cognitive system receives a request which it then parses to extract the major features of the request, which in turn are then used to formulate queries that are applied to the corpus or corpora of data 106. Based on the application of the queries to the corpus or corpora of data 106, a set of hypotheses, or candidate answers/responses to the request, are generated by looking across the corpus or corpora of data 106 for portions of the corpus or corpora of data 106 (hereafter referred to simply as the corpus 106) that have some potential for containing a valuable response to the response. The pipeline 108 of the IBM Watson™ cognitive system then performs deep analysis on the language of the request and the language used in each of the portions of the corpus 106 found during the application of the queries using a variety of reasoning algorithms.

The scores obtained from the various reasoning algorithms are then weighted against a statistical model that summarizes a level of confidence that the pipeline 108 of the IBM Watson™ cognitive system 100, in this example, has regarding the evidence that the potential candidate answer is inferred by the request. This process is be repeated for each of the candidate answers to generate ranked listing of candidate answers which may then be presented to the user that submitted the request, e.g., a user of client computing device 110, or from which a final answer is selected and presented to the user. More information about the pipeline 108 of the IBM Watson™ cognitive system 100 may be obtained, for example, from the IBM Corporation website, IBM Redbooks, and the like. For example, information about the pipeline of the IBM Watson™ cognitive system can be found in Yuan et al., “Watson and Healthcare,” IBM developerWorks, 2011 and “The Era of Cognitive Systems: An Inside Look at IBM Watson and How it Works” by Rob High, IBM Redbooks, 2012.

As noted above, while the input to the cognitive system 100 from a client device may be posed in the form of a natural language question, the illustrative embodiments are not limited to such. Rather, the request may in fact be formatted or structured as any suitable type of request which may be parsed and analyzed using structured and/or unstructured input analysis, including but not limited to the natural language parsing and analysis mechanisms of a cognitive system such as IBM Watson™, to determine the basis upon which to perform cognitive analysis and providing a result of the cognitive analysis. In the case of a healthcare based cognitive system, this analysis may involve processing patient medical records, medical guidance documentation from one or more corpora, and the like, to provide a healthcare oriented cognitive system result.

In the context of the present invention, cognitive system 100 may provide a cognitive functionality for automatically summarizing patient data using medically relevant summarization templates and, if requested, automatically expanding medically relevant summarization templates using semantic expansion. For example, depending upon the particular implementation, the medical information summarization engine based operations may comprise patient electronic medical records (EMRs) evaluation for various purposes, such as for identifying patients that are suitable for a medical trial or a particular type of medical treatment, or the like. Thus, the cognitive system 100 may be a healthcare cognitive system 100 that operates in the medical or healthcare type domains and which may process requests for such healthcare operations via the request processing pipeline 108 input as either structured or unstructured requests, natural language input questions, or the like.” In one illustrative embodiment, the cognitive system 100 is a medical information summarization system that identifies and summarizes the most medically relevant information in a patient's EMRs to meet the needs of the particular medical professional using a medically relevant summarization template with key information identified by the medical professional. Additionally, if requested or directed, the medical information summarization system automatically expands the medically relevant summarization templates using semantic expansion.

As shown in FIG. 1, the cognitive system 100 is further augmented, in accordance with the mechanisms of the illustrative embodiments, to include logic implemented in specialized hardware, software executed on hardware, or any combination of specialized hardware and software executed on hardware, for implementing medical information summarization engine 120. Medical information summarization engine 120 comprises template authorizing engine 122, mapping engine 124, extraction engine 126, matching engine 128, ranking engine 130, presentation engine 132, and expansion engine 134.

In use, a medical professional accesses template authoring engine 122 in which the medical professional provides expectations that the medical professional would like to see in the summary that will eventually be generated by medical information summarization engine 120. For example, if the medical professional is interested in seeing if the patient has ‘Hypertension,’ the medical professional will enter “hypertension” into a ‘Problem List’ category portion of template authoring engine 122. There are multiple ways in which medical professionals may mention hypertension when describing a patient. This may include surface variations such as ‘HYPERTENSION’ or ‘HT’ or ‘HTN’, as well as semantic variations such as ‘High Blood Pressure’ or ‘Hypertensive disease NOS’ or ‘BP+’ etc. However, all of these variations are represented by the same concept and hence a unique identifier (namely ‘C0020538’) in the Unified Medical Language System (UMLS), which is a knowledge base created by the National Library of Medicine. Thus, once the medical professional has input the elements, concepts, terms, parameters, or the like, that the medical professional is interested in, template authoring engine 122 generates a medically relevant summary template identifying which information is to be found from the patient's EMRs and the order in which the information is to be presented. At this point, the medical professional may provide further input to template authoring engine 122 to change which information is to be sought and how the information is to be presented. Once confirmed by the medical professional, template authoring engine 122 generate a medically relevant summary template specifying the expectations of patient information that the medical professional would like to see in a holistic summary of the patient's electronic medical records (EMRs).

With the medically relevant summary template generated, mapping engine 124 maps the free text elements, concepts, terms, parameters, or the like (such as ‘Hypertension’) from the medically relevant summary template to their corresponding unique identifiers in the UMLS, which may be stored as a medical knowledge base, corpus, or the like, as represented by corpus 142. Free text elements may be any form of medical professional generated narratives such as progress notes, radiology reports, discharge summaries, or the like. Mapping engine 124 performs a similar operation on all free text entries in the patient's EMRs, as represented by corpus 140. Based on the mapping of the elements of the medically relevant summary template to medical concepts specified in the medical knowledge base, extraction engine 126 extracts information relevant to the free text elements, concepts, terms, parameters, or the like, from the patient's EMRs 140. Matching engine 128 operates in conjunction with extraction engine 126 to match information extracted by extraction engine 126 to the expected information in the medically relevant summary template. That is, matching engine 128 utilizes the medical knowledge reflected in the medical knowledge base 142 to match the extracted information to both the elements specified in the medically relevant summary template and information in the patient EMRs 140 that is in surrounding portions of the EMRs 140, but is related as indicated by the medical knowledge base 142.

Once matching engine 128 has completed the matching of information, ranking engine 130 ranks the information to be provided in the medically relevant summary of the patient's EMRs with preference being given to the initial specification of expectations made by the medical professional in the medically relevant summary template. That is, a patient may have multiple medical conditions, such as diabetes, hypertension, allergies, asthma, or the like, input into the problem list of the template authoring engine 122. Again, these entries would be subject to the variations that the medical professional chooses to input. Having mapped all medical conditions to unique identifiers of concepts in the knowledge base 142 and performed the extraction and matching of relevant information, ranking engine 130 ranks these problems giving precedence to how closely they match the problems mentioned by the medical professional in the medically relevant summary template.

Thus, following up on the above example, since the problem ‘Hypertension’ is a match with the entries in the summary template, ‘Hypertension’ is ranked the highest when compared with diabetes, allergies, and asthma, which do not match the template. In addition to this direct match for ‘Hypertension’, matching engine 128 would also be able to conclude that although ‘diabetes’ isn't a direct match, it is closely associated with ‘Hypertension’ and hence would be ranked second. This relatedness between diabetes and hypertension may be concluded based on a biomedical knowledge graph. The remaining two problems, namely asthma and allergies, would be ranked last since neither problem is associated with the match ‘Hypertension’. In summary, the problem list (diabetes, hypertension, allergies, asthma) is re-ordered as (hypertension, diabetes, allergies, asthma) since the medical professional mentioned the problem ‘Hypertension’ in the summary template.

Once the ranking is complete, presentation engine 132 generates and presents a holistic summary that may include other extracted patient information that is determined based on the knowledge base to be related, but that is not a direct match to the elements specified in the medically relevant summary template. This other information may be ranked and if sufficiently high enough of a ranking is achieved, i.e. the rank of the information being above a threshold, may be included in the holistic summary of the patient's EMRs. Moreover, the other information may be used to update the medically relevant summary template to include sufficiently high ranking elements from surrounding portions of the patient's EMRs, potentially with the medical professional's approval. In this way, a machine learning of the appropriate elements of a template may be learned and may be tailored to the medical professional. The resulting medically relevant summary template may then be used to extract information for summarizing the EMRs of other patients as well.

The medical professional may also request or indicate that the medically relevant summary template be expanded using semantic expansion, i.e. include semantically relevant terms to those identified by the medical professional in the template authorizing engine 122. If the medical professional makes such a request or indication, then expansion engine 134 operates on the medically relevant summary template by performing synonymous concept identification, related concept identification, and equivalent concept identification, potentially with the use of a medical knowledge base 142. Expansion engine 134 utilizes the identified variants to perform an ontological hierarchical identification process by traversing the medical knowledge base 142 and retrieve all the child/parent concepts of the variants. Expansion engine 134 then adds the variants and the child/parent concepts to the medically relevant summary template thereby forming an expanded medically relevant summary template. Because each of the text elements, concepts, terms, parameters, or the like, from the medically relevant summary template have each have similar variants and/or child/parent concepts, expansion engine 134 operates to mark duplicate text elements, concepts, terms, parameters, or the like, using syntactic and morphological information. Once the marking of the duplicate elements, concepts, terms, parameters, or the like is complete, expansion engine 134 in conjunction with template authorizing engine 122 generates a marked-up expanded medically relevant summary template.

At this point, the medical professional may provide feedback input to template authorizing engine 122 indicating which expanded concepts/terms are correct and which are not for the medical professional's use. Template authorizing engine 122 then feeds back the input from the medical professional to expansion engine 134 in order that expansion engine 134 adjust the operation of this logic when expanding the text elements, concepts, terms, parameters, or the like, for future variants and/or child/parent concepts specified in medically relevant summary template. Thus, in one embodiment, a personalized learning may be provided by medical information summarization engine 120 of related text elements, concepts, terms, parameters, or the like, that is particular to the respective medical professional when generating medically relevant summary templates of patients' EMRs. Once confirmed by the medical professional the process operates as described previously, where mapping engine 124, extraction engine 126, and matching engine 128 operate on the marked-up expanded medically relevant summary template rather than the medically relevant summary template.

In order to provide an example of the operation performed by expansion engine 134, consider, for example, the medical professional will enter “diabetes” into a ‘Problem List’ category portion of template authoring engine 122 with a request or indication that the medically relevant summary template be expanded using semantic expansion. Expansion engine 134 would then perform synonymous concept identification, related concept identification, and equivalent concept identification using the medical knowledge base 142 and identify, for example: diabetes mellitus, mild juvenile diabetes mellitus, diabetes mellitus slow onset, diabetes monitor, diabetes mellitus without complication, diabetes insipidus, diabetes mellitus infantile, diabetes mellitus insulin dependent, diabetes wellbeing questionnaire, diabetes status patient, drug related diabetes mellitus, diabetes mellitus sudden onset, pregnancy induced diabetes, diabetic infant mother syndrome, primary nephrogenic diabetes insipidus, diabetes screen, hypoglycemic event in diabetes, juvenile diabetes mellitus, dm, diabetic peripheral circulatory disorder, diabetic hypoglycemic coma, insulin dependence, high blood sugar, diabetes pregnancy induced, vasopressin resistant diabetes insipidus, unstable diabetes mellitus, neonatal diabetes mellitus, diabetes insulin, diabetes patient education, and gestational diabetes.

Expansion engine 134 utilizes the identified variants to perform an ontological hierarchical identification process by traversing the medical knowledge base 142 and retrieve all the child/parent concepts of the variants, for example: diabetes type 1, diabetes type 2, juvenile diabetes mellitus, diabetes pregnancy induced, gestational diabetes, prediabetes, drug induced diabetes, diabetes mellitus type 1, diabetes mellitus type 2, secondary diabetes mellitus, atypical diabetes mellitus, disorder of glucose metabolism, and disorder of endocrine system. Expansion engine 134 then operates to mark duplicate text elements, concepts, terms, parameters, or the like, using syntactic and morphological information. Thus, expansion engine identifies and marks: gestational diabetes, juvenile diabetes mellitus, and diabetes pregnancy induced.

Accordingly, expansion engine 134 in conjunction with template authorizing engine 122 generates a marked-up expanded medically relevant summary template with a list of text elements, concepts, terms, parameters or the like, including: diabetes mellitus, mild juvenile diabetes mellitus, diabetes mellitus slow onset, diabetes monitor, diabetes mellitus without complication, diabetes insipidus, diabetes mellitus infantile, diabetes mellitus insulin dependent, drug related diabetes mellitus, diabetes mellitus sudden onset, pregnancy induced diabetes, diabetic infant mother syndrome, primary nephrogenic diabetes insipidus, diabetes screen, hypoglycemic event in diabetes, dm, diabetic peripheral circulatory disorder, diabetic hypoglycemic coma, insulin dependence, high blood sugar, diabetes pregnancy induced, vasopressin resistant diabetes insipidus, unstable diabetes mellitus, neonatal diabetes mellitus, diabetes insulin, gestational diabetes, diabetes type 1, diabetes type 2, juvenile diabetes mellitus, prediabetes, drug induced diabetes, diabetes mellitus type 1, diabetes mellitus type 2, secondary diabetes mellitus, atypical diabetes mellitus, disorder of glucose metabolism, and disorder of endocrine system.

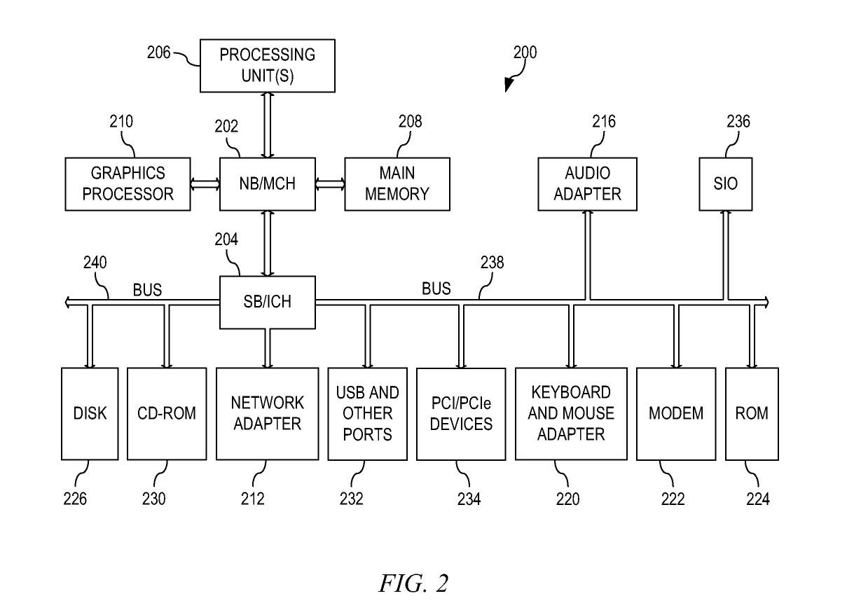

As noted above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. As such, FIG. 2 is provided as an example of one type of data processing system in which aspects of the present invention may be implemented. Many other types of data processing systems may be likewise configured to specifically implement the mechanisms of the illustrative embodiments.

FIG. 2 is a block diagram of an example data processing system in which aspects of the illustrative embodiments are implemented. Data processing system 200 is an example of a computer, such as server 104 or client 110 in FIG. 1, in which computer usable code or instructions implementing the processes for illustrative embodiments of the present invention are located. In one illustrative embodiment, FIG. 2 represents a server computing device, such as a server 104, which, which implements a cognitive system 100 and request processing pipeline 108 augmented to include the additional mechanisms of the illustrative embodiments described hereafter.

In the depicted example, data processing system 200 employs a hub architecture including north bridge and memory controller hub (NB/MCH) 202 and south bridge and input/output (I/O) controller hub (SB/ICH) 204. Processing unit 206, main memory 208, and graphics processor 210 are connected to NB/MCH 202. Graphics processor 210 is connected to NB/MCH 202 through an accelerated graphics port (AGP).

In the depicted example, local area network (LAN) adapter 212 connects to SB/ICH 204. Audio adapter 216, keyboard and mouse adapter 220, modem 222, read only memory (ROM) 224, hard disk drive (HDD) 226, CD-ROM drive 230, universal serial bus (USB) ports and other communication ports 232, and PCI/PCIe devices 234 connect to SB/ICH 204 through bus 238 and bus 240. PCI/PCIe devices may include, for example, Ethernet adapters, add-in cards, and PC cards for notebook computers. PCI uses a card bus controller, while PCIe does not. ROM 224 may be, for example, a flash basic input/output system (BIOS).

HDD 226 and CD-ROM drive 230 connect to SB/ICH 204 through bus 240. HDD 226 and CD-ROM drive 230 may use, for example, an integrated drive electronics (IDE) or serial advanced technology attachment (SATA) interface. Super I/O (SIO) device 236 is connected to SB/ICH 204.

An operating system runs on processing unit 206. The operating system coordinates and provides control of various components within the data processing system 200 in FIG. 2. As a client, the operating system is a commercially available operating system such as Microsoft® Windows 8®. An object-oriented programming system, such as the Java™ programming system, may run in conjunction with the operating system and provides calls to the operating system from Java™ programs or applications executing on data processing system 200.

As a server, data processing system 200 may be, for example, an IBM® eServer™ System p® computer system, running the Advanced Interactive Executive (AIX®) operating system or the LINUX® operating system. Data processing system 200 may be a symmetric multiprocessor (SMP) system including a plurality of processors in processing unit 206. Alternatively, a single processor system may be employed.

Instructions for the operating system, the object-oriented programming system, and applications or programs are located on storage devices, such as HDD 226, and are loaded into main memory 208 for execution by processing unit 206. The processes for illustrative embodiments of the present invention are performed by processing unit 206 using computer usable program code, which is located in a memory such as, for example, main memory 208, ROM 224, or in one or more peripheral devices 226 and 230, for example.

A bus system, such as bus 238 or bus 240 as shown in FIG. 2, is comprised of one or more buses. Of course, the bus system may be implemented using any type of communication fabric or architecture that provides for a transfer of data between different components or devices attached to the fabric or architecture. A communication unit, such as modem 222 or network adapter 212 of FIG. 2, includes one or more devices used to transmit and receive data. A memory may be, for example, main memory 208, ROM 224, or a cache such as found in NB/MCH 202 in FIG. 2.

Those of ordinary skill in the art will appreciate that the hardware depicted in FIGS. 1 and 2 may vary depending on the implementation. Other internal hardware or peripheral devices, such as flash memory, equivalent non-volatile memory, or optical disk drives and the like, may be used in addition to or in place of the hardware depicted in FIGS. 1 and 2. Also, the processes of the illustrative embodiments may be applied to a multiprocessor data processing system, other than the SMP system mentioned previously, without departing from the spirit and scope of the present invention.

Moreover, the data processing system 200 may take the form of any of a number of different data processing systems including client computing devices, server computing devices, a tablet computer, laptop computer, telephone or other communication device, a personal digital assistant (PDA), or the like. In some illustrative examples, data processing system 200 may be a portable computing device that is configured with flash memory to provide non-volatile memory for storing operating system files and/or user-generated data, for example. Essentially, data processing system 200 may be any known or later developed data processing system without architectural limitation.

FIG. 3 is an example diagram illustrating an interaction of elements of a healthcare cognitive system in accordance with one illustrative embodiment. The example diagram of FIG. 3 depicts an implementation of a healthcare cognitive system 300, which may be a cognitive system such as cognitive system 100 described in FIG. 1, that is configured to present contextually relevant patient data in relation to other patients to a medical professional in a graphical user interface. However, it should be appreciated that this is only an example implementation and other healthcare operations may be implemented in other embodiments of the healthcare cognitive system 300 without departing from the spirit and scope of the present invention.

Moreover, it should be appreciated that while FIG. 3 depicts patient 302 and user 306, which may be a medical professional, as human figures, the interactions with and between these entities may be performed using computing devices, medical equipment, and/or the like, such that entities 302 and 306 may in fact be computing devices, e.g., client computing devices. For example, interactions 304, 314, 316, and 330 between patient 302 and user 306 may be performed orally, e.g., a doctor interviewing a patient, and may involve the use of one or more medical instruments, monitoring devices, or the like, to collect information that may be input to the healthcare cognitive system 300. Interactions between user 306 and healthcare cognitive system 300 will be electronic via a user computing device (not shown), such as a client computing device 110 or 112 in FIG. 1, communicating with healthcare cognitive system 300 via one or more data communication links and potentially one or more data networks.

As shown in FIG. 3, in accordance with one illustrative embodiment, a patient 302 presents symptoms 304 of a medical malady or condition to a user 306, such as a healthcare practitioner, technician, or the like. User 306 may interact with patient 302 via a question 314 and response 316 exchange where user 306 gathers more information about patient 302, symptoms 304, and the medical malady or condition of patient 302. It should be appreciated that the requests/responses may in fact also represent user 306 gathering information from patient 302 using various medical equipment, e.g., blood pressure monitors, thermometers, wearable health and activity monitoring devices associated with patient 302 such as a FitBit™, a wearable heart monitor, or any other medical equipment that may monitor one or more medical characteristics of patient 302. In some cases such medical equipment may be medical equipment typically used in hospitals or medical centers to monitor vital signs and medical conditions of patients that are present in hospital beds for observation or medical treatment.

In response, user 306 submits request 308 to healthcare cognitive system 300, such as via a user interface on a client computing device that is configured to allow users to submit requests to healthcare cognitive system 300 in a format that healthcare cognitive system 300 is able to parse and process. Request 308 may include, or be accompanied with, area of interest 318. The area of interest 318 may include, for example, elements, concepts, terms, parameters or the like, to retrieve from the patient's EMRs 322 for patient 302. Any information about patient 302 that may be relevant to a cognitive evaluation of patient 302 by healthcare cognitive system 300 may be included in request 308 and/or area of interest 318.

Healthcare cognitive system 300 provides a cognitive system that is specifically configured to perform an implementation specific healthcare oriented cognitive operation. In the depicted example, this cognitive medical treatment recommendation operation is directed to automatically summarizing patient data associated with patient 302 from patient EMRs 322 using medically relevant summarization templates and providing a holistic summary 328 of patient 302 associated with the area of interest to user 306 and to automatically expanding medically relevant summarization templates using semantic expansion, i.e. include semantically relevant terms to those identified by the user 306. Healthcare cognitive system 300 operates on request 308 utilizing information gathered from medical corpus and other source data 326, treatment guidance data 324, and patient EMRs 322 associated with patient 302 to generate holistic summary 328. Holistic summary 328 may be presented with associated supporting evidence, obtained from data sources 322, 324, and 326, indicating the reasoning as to why the holistic summary 328 is being provided.

For example, based on request 308 and area of interest 318, healthcare cognitive system 300 may operate on the request to parse request 308 and area of interest 318 to determine what is being requested and the criteria upon which the request is to be generated as identified by area of interest 318, and may perform various operations for generating queries that are sent to the data sources 322, 324, and 326 to retrieve data, generate associated indications associated with the data, and provides supporting evidence found in the data sources 322, 324, and 326. In the depicted example, patient EMRs 322 is a patient information repository that collects patient data from a variety of sources, e.g., hospitals, laboratories, physicians' offices, health insurance companies, pharmacies, etc. Patient EMRs 322 store various information about individual patients, such as patient 302, in a manner (structured, unstructured, or a mix of structured and unstructured formats) that the information may be retrieved and processed by healthcare cognitive system 300. This patient information may comprise various demographic information about patients, personal contact information about patients, employment information, health insurance information, laboratory reports, physician reports from office visits, hospital charts, historical information regarding previous diagnoses, symptoms, treatments, prescription information, etc. Based on an identifier of the patient 302, the patient's corresponding EMRs 322 from this patient repository may be retrieved by healthcare cognitive system 300 and searched/processed to generate holistic summary 328.

Treatment guidance data 324 provides a knowledge base of medical knowledge that is used to identify potential treatments for a patient's medical condition based on area of interest 318 and historical information presented in patient's EMRs 322. Treatment guidance data 324 may be obtained from official treatment guidelines and policies issued by medical authorities, e.g., the American Medical Association, may be obtained from widely accepted physician medical and reference texts, e.g., the Physician's Desk Reference, insurance company guidelines, or the like. The treatment guidance data 324 may be provided in any suitable form that may be ingested by the healthcare cognitive system 300 including both structured and unstructured formats.

In some cases, such treatment guidance data 324 may be provided in the form of rules that indicate the criteria required to be present, and/or required not to be present, for the corresponding treatment to be applicable to a particular patient for treating a particular symptom or medical malady/condition. For example, the treatment guidance data 324 may comprise a treatment recommendation rule that indicates that for a treatment of Decitabine, strict criteria for the use of such a treatment is that patient 302 is less than or equal to 60 years of age, has acute myeloid leukemia (AML), and no evidence of cardiac disease. Thus, for a patient 302 that is 59 years of age, has AML, and does not have any evidence in their area of interest 318 or patient EMRs 322 indicating evidence of cardiac disease, the following conditions of the treatment rule exist:
Age<=60 years=59 (MET);
Patient has AML=AML (MET); and
Cardiac Disease=false (MET)
   Since all of the criteria of the treatment rule are met by the specific information about this patient 302, then the treatment of Decitabine is a candidate treatment recommendation for consideration for this patient 302. However, if the patient had been 69 years old, the first criterion would not have been met and the Decitabine treatment would not be a candidate treatment recommendation for consideration for this patient 302. Various potential treatment recommendations may be evaluated by healthcare cognitive system 300 based on ingested treatment guidance data 324 to identify subsets of candidate treatment recommendations for further consideration by healthcare cognitive system 300 by identifying such candidate treatment recommendations based on evidential data obtained from patient EMRs 322 and medical corpus and other source data 326.

For example, data mining processes may be employed to mine the data in sources 322 and 326 to identify evidential data supporting and/or refuting the applicability of the candidate treatment recommendations to the particular patient 302 as characterized by the area of interest 318 and EMRs 322. For example, for each of the criteria of the treatment rule, the results of the data mining provides a set of evidence that supports giving the treatment in the cases where the criterion is “MET” and in cases where the criterion is “NOT MET.” Healthcare cognitive system 300 processes the evidence in accordance with various cognitive logic algorithms to generate an indicator for each candidate treatment recommendation indicating a confidence that the corresponding candidate treatment recommendation is valid for patient 302. The candidate treatment recommendations may then be presented to user 306 as a listing of holistic summary 328. Holistic summary 328 may be presented to user 306 in a manner that the underlying evidence evaluated by healthcare cognitive system 300 may be accessible, such as via a drilldown interface, so that user 306 may identify the reasons why holistic summary 328 is being provided by healthcare cognitive system 300.

In accordance with the illustrative embodiments herein, healthcare cognitive system 300 is augmented to include medical information summarization engine 340. Medical information summarization engine 340 comprises template authorizing engine 342, mapping engine 344, extraction engine 346, matching engine 348, ranking engine 350, presentation engine 352, and expansion engine 354. In use, user 306 accesses template authoring engine 122 in which the user 306 provides request 308 and area of interest 318 that user 306 would like to see in holistic summary 328 that will eventually be generated by medical information summarization engine 340. For example, if user 306 is interested in seeing if patient 302 has ‘Hypertension,’ user 306 enters “hypertension” into a ‘Problem List’ category portion of template authoring engine 342. There are multiple ways in user 306 may mention hypertension when describing patient 302. This may include surface variations such as ‘HYPERTENSION’ or ‘HT’ or ‘HTN’, as well as semantic variations such as ‘High Blood Pressure’ or ‘Hypertensive disease NOS’ or ‘BP+’ etc. However, all of these variations are represented by the same concept and hence a unique identifier (namely ‘C0020538’) in the Unified Medical Language System (UMLS), which is a knowledge base created by the National Library of Medicine.

Thus, once user 306 has input area of interest 318 through elements, concepts, terms, parameters, or the like, that user 306 is interested in, template authoring engine 342 generates a medically relevant summary template identifying which information is to be found from the EMRs of patient 302 stored in patient EMRs 322 and the order in which the information is to be presented. At this point, user 306 may provide further input to template authoring engine 342 to change which information is to be sought and how the information is to be presented. Once confirmed by user 306, template authoring engine 342 generate a medically relevant summary template specifying the expectations of patient information that user 306 would like to see in a holistic summary of EMRs of patient 302 stored in patient EMRs 322.

With the medically relevant summary template generated, mapping engine 344 maps the free text elements, concepts, terms, parameters, or the like (such as ‘Hypertension’) from the medically relevant summary template to their corresponding unique identifiers in the UMLS, which may be stored in medical corpus and other source data 326. Mapping engine 344 performs a similar operation on all free text entries in the EMRs of patient 302 stored in patient EMRs 322. Based on the mapping of the elements of the medically relevant summary template to medical concepts specified in medical corpus and other source data 326, extraction engine 346 extracts information relevant to the free text elements, concepts, terms, parameters, or the like, from the EMRs of patient 302. Matching engine 348 operates in conjunction with extraction engine 346 to match information extracted by extraction engine 346 to the expected information in the medically relevant summary template. That is, matching engine 348 utilizes the medical knowledge reflected in the medical corpus and other source data 326 to match the extracted information to both the elements specified in the medically relevant summary template and information in the EMRs of patient 302 that is in surrounding portions of the EMRs, but is related as indicated by medical corpus and other source data 326.

Once matching engine 348 has completed the matching of information, ranking engine 350 ranks the information to be provided in the holistic summary of the patient's EMRs with preference being given to the initial specification of expectations made by user 306 in the medically relevant summary template. That is, patient 302 may have multiple medical conditions, such as diabetes, hypertension, allergies, asthma, or the like, input into the problem list of the template authoring engine 342. Again, these entries would be subject to the variations that user 306 chooses to input. Having mapped all medical conditions to unique identifiers of concepts in the medical corpus and other source data 326 and performed the extraction and matching of relevant information, ranking engine 350 ranks these problems giving precedence to how closely they match the problems mentioned by user 306 in the medically relevant summary template.

Thus, following up on the above example, since the problem ‘Hypertension’ is a match with the entries in the summary template, ‘Hypertension’ is ranked the highest when compared with diabetes, allergies, and asthma, which do not match the template. In addition to this direct match for ‘Hypertension’, matching engine 348 would also be able to conclude that although ‘diabetes’ isn't a direct match, it is closely associated with ‘Hypertension’ and hence would be ranked second. The remaining two problems, namely asthma and allergies, would be ranked last since neither problem is associated with the match ‘Hypertension’. In summary, the problem list (diabetes, hypertension, allergies, asthma) is re-ordered as (hypertension, diabetes, allergies, asthma) since user 306 mentioned the problem ‘Hypertension’ in the summary template.

Once the ranking is complete, presentation engine 352 generates and presents a holistic summary that may include other extracted patient information that is determined based on the knowledge base to be related, but that is not a direct match to the elements specified in the medically relevant summary template. This other information may be ranked and if sufficiently high enough of a ranking is achieved, may be included in the holistic summary of the patient's EMRs. Moreover, this information may be used to update the medically relevant summary template to include sufficiently high ranking elements from surrounding portions of the patient's EMRs, potentially with approval from user 306. In this way, a machine learning of the appropriate elements of a template may be learned and may be tailored to user 306. The resulting medically relevant summary template may then be used to extract information for summarizing the EMRs of other patients as well.

Therefore, the illustrative embodiments provide mechanisms that automatically summarize patient data using medically relevant summarization templates. The mechanisms distill important information from patient's EMRs 322 using an expert verified summarization template. The mechanisms create a summary template that describes key information identified by the medical professional to be fetched from patient's EMRs 322. The mechanisms aggregate redundant pieces of information for conciseness and extract patient information from the patient's EMRs 322 that matches the summarization template. The mechanisms then rank the extracted patient information from the patient's EMRs 322 in light of those matches and generate a holistic summary 328 that summarizes the most salient portions of the patient's EMRs 322 for use by user 306 in making a medical decision regarding patient 302.

User 306 may also request or indicate that the medically relevant summary template be expanded using semantic expansion, i.e. include semantically relevant terms to those identified by user 306 in the template authorizing engine 342. If user 306 makes such a request or indication, then expansion engine 354 operates on the medically relevant summary template by performing synonymous concept identification, related concept identification, and equivalent concept identification, potentially with the use of medical corpus and other source data 326. Expansion engine 354 utilizes the identified variants to perform an ontological hierarchical identification process by traversing medical corpus and other source data 326 and retrieve all the child/parent concepts of the variants. Expansion engine 354 then adds the variants and the child/parent concepts to the medically relevant summary template thereby forming an expanded medically relevant summary template. Because each of the text elements, concepts, terms, parameters, or the like, from the medically relevant summary template have each have similar variants and/or child/parent concepts, expansion engine 354 operates to mark duplicate text elements, concepts, terms, parameters, or the like, using syntactic and morphological information. Once the marking of the duplicate text elements, concepts, terms, parameters, or the like is complete, expansion engine 354 in conjunction with template authorizing engine 342 generates a marked-up expanded medically relevant summary template.

At this point, user 306 may provide feedback input to template authorizing engine 342 indicating which expanded concepts/terms are correct and which are not for use by user 306. Template authorizing engine 342 then feeds back the input from user 306 to expansion engine 354 in order that expansion engine 354 adjust the operation of this logic when expanding the text elements, concepts, terms, parameters, or the like, for future variants and/or child/parent concepts specified in medically relevant summary template. Thus, in one embodiment, a personalized learning may be provided by medical information summarization engine 340 of related text elements, concepts, terms, parameters, or the like, that is particular to the respective user 306 when generating medically relevant summary templates of patients' EMRs. Once confirmed by user 306 the process operates as described previously, where mapping engine 344, extraction engine 346, and matching engine 348 operate on the marked-up expanded medically relevant summary template rather than the medically relevant summary template.

Thus, the illustrative embodiments provide mechanisms that automatically expand medically relevant summarization templates using semantic expansion. In the creation of the summary template that describes key information identified by user 306 to be fetched from patient's EMRs 322, user 306 may request or indicate that the summary template be expanded to include semantically relevant terms to those identified by user 306. Thus, the mechanisms identify the seed concepts and terms provided by user 306. The mechanisms expand the seed concepts and terms by identifying medical variants and related concepts based on an ontological hierarchy and biomedical knowledge graph. In identifying the medical variants and related concepts of the seed concepts and terms duplicates concepts may be identified. Thus, the mechanisms also mark duplicate concepts in creating the marked-up expanded summarization template. The mechanisms then present the marked-up expanded medically relevant summarization template to user 306 prior to summarizing patient data from the patient's EMRs 322 using the marked-up expanded medically relevant summarization templates.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

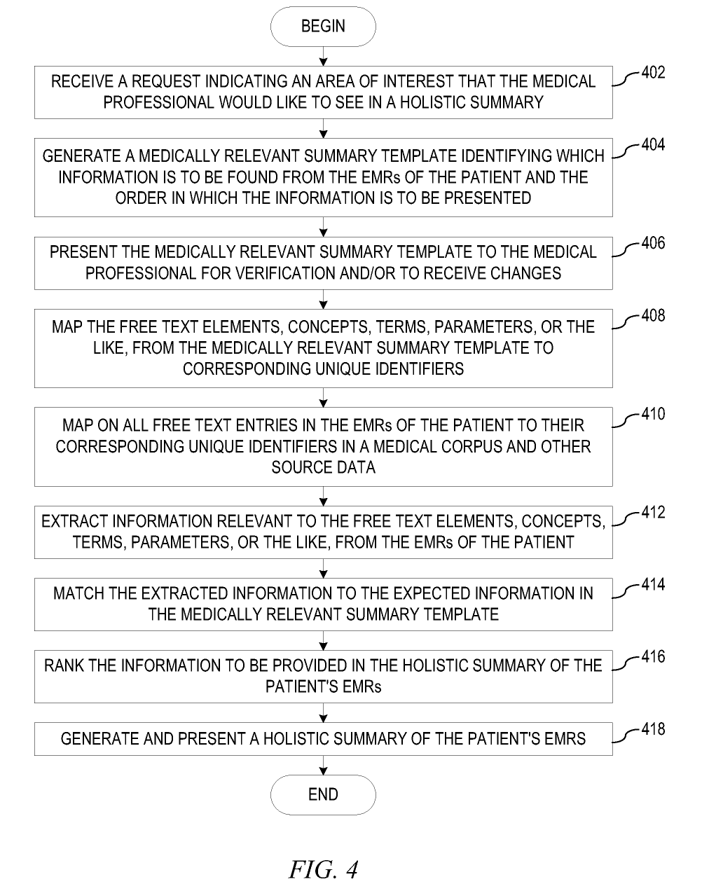

FIG. 4 depicts a functional block diagram of operations performed by a medical information summarization engine in automatically summarizing patient data using medically relevant summarization templates in accordance with an illustrative embodiment. As the operation begins, the medical information summarization engine receives a request indicating an area of interest that the medical professional would like to see in a holistic summary (step 402). The medical information summarization engine generates a medically relevant summary template identifying which information is to be found from the EMRs of the patient and the order in which the information is to be presented (step 404). The medical information summarization engine may present the medically relevant summary template to the medical professional for verification and/or to receive changes to which information is to be sought and how the information is to be presented in the holistic summary (step 406). Once confirmed by the medical professional, the medical information summarization engine maps the free text elements, concepts, terms, parameters, or the like, from the medically relevant summary template to their corresponding unique identifiers in a medical corpus and other source data, such as a Unified Medical Language System (UMLS) (step 408).

The medical information summarization engine also performs a mapping on all free text entries in the EMRs of the patient to their corresponding unique identifiers in a medical corpus and other source data (step 410). Based on the mapping of the elements of the medically relevant summary template to medical concepts specified in the medical corpus and other source data, the medical information summarization engine extracts information relevant to the free text elements, concepts, terms, parameters, or the like, from the EMRs of the patient (step 412). The medical information summarization engine then matches the extracted information to the expected information in the medically relevant summary template (step 414). That is, the medical information summarization engine utilizes the medical knowledge reflected in the medical corpus and other source data to match the extracted information to both the elements specified in the medically relevant summary template and information in the EMRs of the patient that is in surrounding portions of the EMRs, but is related as indicated by the medical corpus and other source data.

Once the medical information summarization engine has completed the matching of information, the medical information summarization engine ranks the information to be provided in the holistic summary of the patient's EMRs with preference being given to the initial specification of expectations made by the medical professional in the medically relevant summary template (step 416). Once the ranking is complete, the medical information summarization engine generates and presents a holistic summary of the patient's EMRs that may include other extracted patient information that is determined based on the knowledge base to be related, but that is not a direct match to the elements specified in the medically relevant summary template (step 418). This other information may be ranked and, if sufficiently high enough of a ranking is achieved, may be included in the holistic summary of the patient's EMRs. Moreover, this information may be used to update the medically relevant summary template to include sufficiently high ranking elements from surrounding portions of the patient's EMRs, potentially with approval from the medical professional. In this way, a machine learning of the appropriate elements of a template may be learned and may be tailored to medical professional. The resulting medically relevant summary template may then be used to extract information for summarizing the EMRs of other patients as well. The operation terminates thereafter.

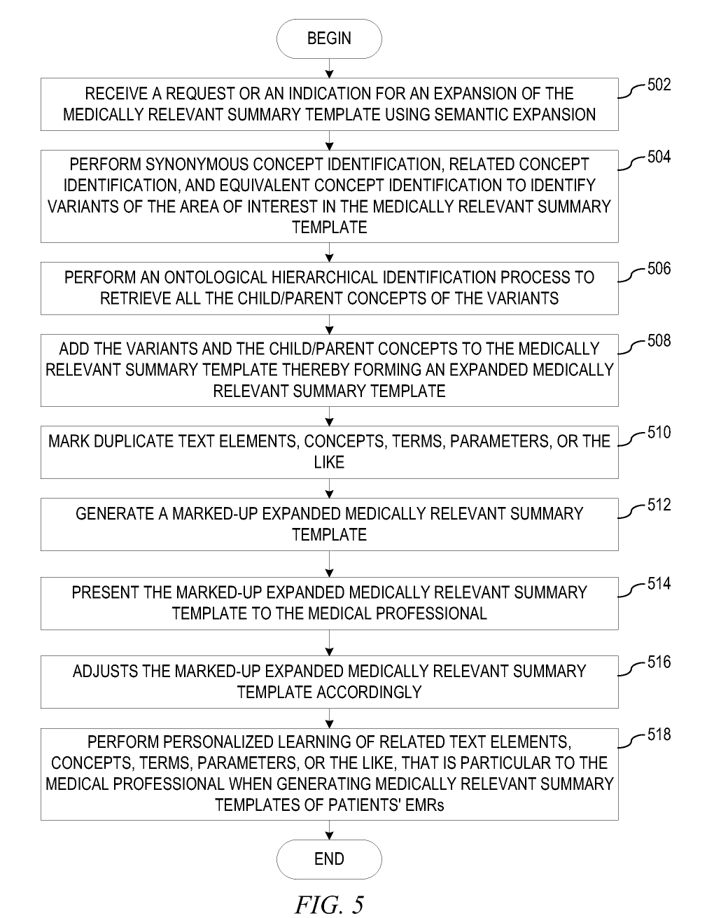

FIG. 5 depicts a functional block diagram of operations performed by a medical information summarization engine in automatically expand medically relevant summarization templates using semantic expansion in accordance with an illustrative embodiment. As the operation begins, the medical information summarization engine receives a request or an indication for an expansion of the medically relevant summary template using semantic expansion, i.e. include semantically relevant terms to those identified by the medical professional (step 502). If the medical professional makes such a request or indication, then the medical information summarization engine operates on the medically relevant summary template by performing synonymous concept identification, related concept identification, and equivalent concept identification, potentially with the use of the medical corpus and other source data to identify variants of the free text elements, concepts, terms, parameters, or the like, provided by the medical professional (step 504).

The medical information summarization engine utilizes the identified variants to perform an ontological hierarchical identification process by traversing the medical corpus and other source data and retrieve all the child/parent concepts of the variants (step 506). The medical information summarization engine adds the variants and the child/parent concepts to the medically relevant summary template thereby forming an expanded medically relevant summary template (step 508). Because each of the text elements, concepts, terms, parameters, or the like, from the medically relevant summary template have each have similar variants and/or child/parent concepts, the medical information summarization engine marks duplicate text elements, concepts, terms, parameters, or the like, using syntactic and morphological information (step 510). Once the marking of the duplicate elements, concepts, terms, parameters, or the like is complete, the medical information summarization engine generates a marked-up expanded medically relevant summary template (step 512).

The medical information summarization engine then presents the marked-up expanded medically relevant summary template to the medical professional so that the medical professional may provide feedback input indicating which expanded concepts/terms are correct and which are not for use by the medical professional (step 514). If feedback input is provided, the medical information summarization engine adjusts the marked-up expanded medically relevant summary template accordingly (step 516). The medical information summarization engine also utilizes the feedback input as well as the final version of the marked-up expanded medically relevant summary template to perform personalized learning of related text elements, concepts, terms, parameters, or the like, that is particular to the medical professional when generating medically relevant summary templates of patients' EMRs (step 518). Once confirmed by the medical professional the process operates as described previously with regard to FIG. 4 utilizing the marked-up expanded medically relevant summary template rather than the medically relevant summary template. The operation terminates thereafter.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Thus, the illustrative embodiments provide mechanisms for automatically summarizing patient data using medically relevant summarization templates. The mechanisms create a summary template that describes key information identified by the medical professional to be fetched from the patient's EMRs. The mechanisms aggregate redundant pieces of information for conciseness and extract patient information from the patient's EMRs that matches the summarization template. The mechanisms then rank the extracted patient information from the patient's EMRs in light of those matches and generate a patient EMR summary output that summarizes the most salient portions of the patient's EMRs for use by the medical professional in making a medical decision regarding the patient, based on the ranking of the patient information.

Additionally, the illustrative embodiments provide mechanisms for automatically expanding medically relevant summarization templates using semantic expansion. In the creation of the summary template that describes key information identified by the medical professional to be fetched from the patient's EMRs, the medical professional may request or indicate that the summary template be expanded to include semantically relevant terms to those identified by the medical professional. Thus, the mechanisms identify the seed concepts and terms provided by the medical professional. The mechanisms expand the seed concepts and terms by identifying medical variants and related concepts based on an ontological hierarchy and biomedical knowledge graph. In identifying the medical variants and related concepts of the seed concepts and terms duplicates concepts may be identified. Thus, the mechanisms also mark duplicate concepts in creating a marked-up expanded summarization template. The mechanisms then present a marked-up expanded medically relevant summarization template that is presented to the medical professional prior to summarizing patient data from the patient's EMRs using the marked-up expanded medically relevant summarization templates.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising a processor and a memory, the memory comprising instructions that are executed by the processor to specifically configure the processor to implement a medical information summarization engine (MISE), the method comprising:
receiving, by the MISE executing in the data processing system, input specifying a summarization template, wherein the summarization template specifies terms or concepts of interest to a medical professional when making a medical decision regarding a patient;
expanding, by the MISE, the summarization template based on related concepts or related terms that are related to the terms or concepts of interest specified in the summarization template, to thereby generate an expanded summarization template, wherein the expansion operation expands the summarization template based on a traversal of a medical knowledge base of the related terms or relate concepts;
receiving, by the MISE, electronic medical records (EMR) for a patient;
processing, by the MISE, the EMR of the patient based on the expanded summarization template to extract patient information corresponding to the terms or concepts of interest and the related concepts or related terms; and
generating and outputting, by the MISE, a holistic summary of the EMR of the patient that summarizes the most salient portions of the patient EMR for use by the medical professional in making the medical decision regarding the patient, based on extracted patient information obtained from processing the patient EMR based on the expanded summarization template.

2. The method of claim 1, wherein the expansion operation expands the summarization template based on a traversal of a medical knowledge base of the related terms or related concepts.

3. The method of claim 1, wherein expanding the summarization template further comprises:
performing, by the MISE, concept identification to identify variants of the terms and concepts of interest to the medical professional;
utilizing the variants, performing, by the MISE, an ontological hierarchical identification process by traversing the medical knowledge base to retrieve all the child/parent concepts of the variants; and
adding, by the MISE, the variants and the child/parent concepts to the summarization template thereby forming an expanded summarization template.

4. The method of claim 3, wherein the concept identification comprises at least one of synonymous concept identification, related concept identification, and equivalent concept identification.

5. The method of claim 3, further comprising:
marking, by the MISE, duplicate terms or concepts using syntactic and morphological information.

6. The method of claim 1, further comprising:
prior to processing the EMR of the patient, presenting, by the MISE, the expanded summarization template to the medical professional; and
responsive to the medical professional changing the expanded summarization template, adjusting, by the MISE, the expanded summarization template.

7. The method of claim 1, further comprising:
adjusting, by the MISE, the summarization template according to the expanded summarization template.

8. A computer program product comprising a computer readable storage medium having a computer readable program stored therein, wherein the computer readable program, when executed on a computing device, causes the computing device to implement a medical information summarization engine (MISE) which operates to:
receive input specifying a summarization template, wherein the summarization template specifies terms or concepts of interest to a medical professional when making a medical decision regarding a patient;
expand the summarization template based on related concepts or related terms that are related to the terms or concepts of interest specified in the summarization template, to thereby generate an expanded summarization template, wherein the expansion operation expands the summarization template based on a traversal of a medical knowledge base of the related terms or relate concepts;
receive electronic medical records (EMR) for a patient;
process the EMR of the patient based on the expanded summarization template to extract patient information corresponding to the terms or concepts of interest and the related concepts or related terms; and
generate and output a holistic summary of the EMR of the patient that summarizes the most salient portions of the patient EMR for use by the medical professional in making the medical decision regarding the patient, based on extracted patient information obtained from processing the patient EMR based on the expanded summarization template.

9. The computer program product of claim 8, wherein the expansion operation expands the summarization template based on a traversal of a medical knowledge base of the related terms or related concepts.

10. The computer program product of claim 8, wherein the computer readable program to expand the summarization template further causes the computing device to implement the MISE which operates to:
perform concept identification to identify variants of the terms and concepts of interest to the medical professional;
utilizing the variants, perform an ontological hierarchical identification process by traversing the medical knowledge base to retrieve all the child/parent concepts of the variants; and
add the variants and the child/parent concepts to the summarization template thereby forming an expanded summarization template.

11. The computer program product of claim 10, wherein the concept identification comprises at least one of synonymous concept identification, related concept identification, and equivalent concept identification.

12. The computer program product of claim 10, wherein the computer readable program further causes the computing device to implement the MISE which operates to:
mark duplicate terms or concepts using syntactic and morphological information.

13. The computer program product of claim 8, wherein the computer readable program further causes the computing device to implement the MISE which operates to:
prior to processing the EMR of the patient, present the expanded summarization template to the medical professional; and
responsive to the medical professional changing the expanded summarization template, adjust the expanded summarization template.

14. The computer program product of claim 8, wherein the computer readable program further causes the computing device to implement the MISE which operates to:
adjust the summarization template according to the expanded summarization template.

15. An apparatus comprising:
a processor; and
a memory coupled to the processor, wherein the memory comprises instructions which, when executed by the processor, cause the processor to implement a medical information summarization engine (MISE) that operates to:
receive input specifying a summarization template, wherein the summarization template specifies terms or concepts of interest to a medical professional when making a medical decision regarding a patient;
expand the summarization template based on related concepts or related terms that are related to the terms or concepts of interest specified in the summarization template, to thereby generate an expanded summarization template, wherein the expansion operation expands the summarization template based on a traversal of a medical knowledge base of the related terms or relate concepts;
receive electronic medical records (EMR) for a patient;
process the EMR of the patient based on the expanded summarization template to extract patient information corresponding to the terms or concepts of interest and the related concepts or related terms; and
generate and output a holistic summary of the EMR of the patient that summarizes the most salient portions of the patient EMR for use by the medical professional in making the medical decision regarding the patient, based on extracted patient information obtained from processing the patient EMR based on the expanded summarization template.

16. The apparatus of claim 15, wherein the expansion operation expands the summarization template based on a traversal of a medical knowledge base of the related terms or related concepts.

17. The apparatus of claim 15, wherein the instructions to expand the summarization template further cause the processor to implement the MISE which operates to:
perform concept identification to identify variants of the terms and concepts of interest to the medical professional;
utilizing the variants, perform an ontological hierarchical identification process by traversing the medical knowledge base to retrieve all the child/parent concepts of the variants; and
add the variants and the child/parent concepts to the summarization template thereby forming an expanded summarization template.

18. The apparatus of claim 17, wherein the concept identification comprises at least one of synonymous concept identification, related concept identification, and equivalent concept identification.

19. The apparatus of claim 17, wherein the instructions further cause the processor to implement the MISE which operates to:
mark duplicate terms or concepts using syntactic and morphological information.

20. The apparatus of claim 15, wherein the instructions further cause the processor to implement the MISE which operates to:
prior to processing the EMR of the patient, present the expanded summarization template to the medical professional; and
responsive to the medical professional changing the expanded summarization template, adjust the expanded summarization template.
